(19) United States
(12) Patent Application Publication
(10) Pub. No.: US 2005/0272824 A1
(43) Pub. Date: Dec. 8, 2005

(54) Methods for monitoring combretastatin resistance

(76) Inventors: Kevin G. Pinney, Woodway, TX (US); Hania Wehbe, Hewitt, TX (US); Christopher M. Kearney, Waco, TX (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/089,612

(22) Filed: Mar. 25, 2005

Related U.S. Application Data
(60) Provisional application No. 60/557,070, filed on Mar. 26, 2004.

(51) Int. Cl. A61K 31/075; G01N 33/574

(52) U.S. Cl. 514/720; 435/7.23

(57) ABSTRACT

Methods and diagnostic kits for monitoring combretastatin resistance are provided.

### RELATED APPLICATIONS

This application claims the benefit of prior-filed provisional patent application U.S. Ser. No. 60/557,070, filed Mar. 26, 2004, entitled “Novel Combretastatin Analogs: Potential Agents of Tubulin and Vascular Targeting”. The entire content of the above-referenced application is incorporated herein by reference.

### BACKGROUND OF THE INVENTION

Cellular transformation during the development of cancer involves multiple alterations in the normal pattern of cell growth regulation. Primary events in the process of carcinogenesis involve the activation of oncogene function by some means (e.g., amplification, mutation, chromosomal rearrangement), and in many cases, the removal of anti-oncogene function. In the most malignant and untreatable tumors, normal restraints on cell growth are completely lost as transformed cells escape from their primary sites and metastasize to other locations in the body. One reason for the enhanced growth and invasive properties of some tumors may be the acquisition of increasing numbers of mutations in oncogenes, with cumulative effect (Bear, et al., Proc. Natl. Acad. Sci. USA 86:7495-7499, (1989)).

Alternatively, insofar as oncogenes function through the normal cellular signaling pathways required for organismal growth and cellular function (reviewed in McCormick, et al., Nature, 63:15-16, (1993)), additional alterations in the oncogenic signaling pathways may also contribute to tumor malignancy (Gilks, et al., Mol. Cell Biol. 13:1759-1768, (1993)), even though mutations in the signaling pathways alone may not cause cancer.

Several discrete classes of proteins are known to be involved in bringing about the different types of changes in cell division properties and morphology associated with transformation. These changes can be summarized as, first, the promotion of continuous cell cycling (immortalization); second, the loss of responsiveness to growth inhibitory signals and cell apoptotic signals; and third, the morphological restructuring of cells to enhance invasive properties.

The National Cancer Institute has estimated that in the United States alone, 1 in 3 people will be struck with cancer during their lifetime. Moreover, approximately 50% to 60% of people contracting cancer will eventually succumb to the disease. The widespread occurrence of this disease underscores the need for improved anticancer regimens for the treatment of malignancy.

Due to the wide variety of cancers presently observed, numerous anticancer agents have been developed to destroy cancer within the body. These compounds are administered to cancer patients with the objective of destroying or otherwise inhibiting the growth of malignant cells while leaving normal, healthy cells undisturbed. Unfortunately, deleterious side effects are associated with these agents. For example, fluorouracil, a commonly used antineoplastic agent causes swelling or redness of normal skin, black or tarry stools, blood in the urine, chest pain, confusion, diarrhea, shortness of breath, and drowsiness. Administration of fluorouracil has also been associated with fever, chills, cough, sore throat, lower back pain, mouth sores, nausea, vomiting, pain and/or difficulty passing urine. Taxanes, mitotic inhibitors which are commonly used for anti-cancer use, have been associated with cardiovascular events such as syncope, rhythm abnormalities, hypertension and venous thrombosis; bone marrow suppression, neutropenia, anemia, peripheral neuropathy arthralgia/myalgia, nausea/vomiting and alopecia, to name only a few.

In addition to their often considerable toxicity, many conventional anticancer agents are ineffective or gradually fail to be effective in treating certain tumors due to the presence of acquired or intrinsic tumor mutations that confer resistance to the chemotherapeutic. Acquired or intrinsic drug resistance is a major complication in cancer chemotherapy and accounts for the failure of chemotherapy to cure the majority of cancer patients (Gottesman et al., Annu Rev. Biochem., 62:385-427 (1993); Van Der Zee, et al., Gynecologic Oncol., 58:165-178 (1995); Casazza et al., Cancer Treat. Res. 87:1-171, (1996)). For example, tumors may acquire resistance to platinum coordination compounds such as cisplatin due to their acquisition of mutations which cause a decreased intracellular accumulation of cisplatin or increased DNA repair (Chu et al., J. Biol. Chem., 269: 787-790, (1994)). Drug resistance also has significant clinical implications. When cells become resistant to a particular anticancer agent, the doses must be increased, leading to a worsening of drug-associated toxicities.

Combretastatins are another class of anticancer agents. Combretastatins have been isolated from stem wood of the African tree Combretum caffrum (Combretaceae), and are potent inhibitors of microtubulin assembly. Combretastatin A-4 (“CA4”) is significantly active against the US National Cancer Institute's (NCI) murine L1210 and P338 lymphocytic leukemia cell lines. In addition, CA4 was found to compete with combretastatin A-1 (“CA1”), another compound isolated from Combretum caffrum, as a potent inhibitor of colchicine binding to tubulin. CA4 also strongly retards the growth of certain cell lines (ED₅₀<0.01 (g/ml)) and is a powerful anti-mitotic agent. See, e.g., U.S. Pat. No. 4,996,237. Since the solubility of the combretastatins is very limited, prodrugs have been developed, such as combretastatin A-4 phosphate and combretastatin A-1 diphosphate (hereinafter “CA4P” and “CA1P” respectively), to increase the solubility, and thus the efficacy of CA-4 and CA-1.

Although CA4P and CA1P have activity as inhibitors of tumor cell proliferation, their primary mechanism of action has been shown to be one of “vascular targeting”, in which the neovasculature of solid tumors is selectively disrupted, resulting in a transient decrease or complete shutdown of tumor blood flow that results in secondary tumor cell death due to hypoxia, acidosis, and/or nutrient deprivation (Dark et al., Cancer Res., 57: 1829-34, (1997); Chaplin et al., Anticancer Res., 19: 189-96, (1999); Hill et al., Anticancer Res., 22(3):1453-8 vast majority of the tumor mass, some tumors are nonetheless resistant to treatment with combretastatins.

### SUMMARY OF THE INVENTION

The present invention addresses a need in the art for methods of monitoring or prognosticating resistance or susceptibility of a patient to anticancer agents such as combretastatins, so that appropriate and effective therapy can be tailored to the patient's needs.

The present invention provides methods of monitoring or prognosticating resistance or susceptibility of a patient to tubulin depolymerizing anticancer agents, in particular combretastatins, comprising obtaining a biological sample from the patient and analyzing the sample for a profile of tubulin expression.

In one aspect, the invention provides methods for determining the clinical prognosis of a patient suffering from cancer, wherein said patient has been administered a combretastatin, the method comprising: (a) obtaining a biological sample from the patient; (b) determining a tubulin isotype level of the biological sample; (c) comparing the tubulin isotype level with a baseline level; (d) correlating the tubulin isotype level with an indication of unfavorable prognosis if the tubulin isotype level is lower than the baseline level or correlating the tubulin isotype level with an indication of favorable prognosis if the tubulin isotype level is higher than the baseline level.

In another aspect, the invention provides methods for selecting a patient for further treatment with a combretastatin, the method comprising: (a) determining a tubulin isotype level in a first biological sample from the patient; (b) administering the combretastatin to the patient; (c) determining a second tubulin isotype level from a second biological sample obtained from the patient; (d) comparing the first and second tubulin isotype levels; and (d) electing the patient for further treatment if an increase in tubulin isotype level is observed.

In another aspect, the invention provides a method for monitoring the progression of a tumor in patient, the method comprising: (a) determining a tubulin isotype level in a first biological sample from the patient; (b) administering the combretastatin to the patient; (c) determining a second tubulin isotype level from a second biological sample obtained from the patient; and (d) comparing the first and second tubulin isotype levels.

In certain embodiments, the biological sample is a tumor cell. In another embodiment, the biological sample is an endothelial cell.

In one embodiment, the tubulin isotype is tubulin isotype III. In another embodiment, the tubulin isotype is a tubulin isotype IV.

In another aspect, the invention provides a diagnostic kit for profiling isotype expression in a patient comprising a probe for detecting tubulin isotype expression in a biological sample obtained from the patient.

### BRIEF DESCRIPTION OF THE DRAWINGS

FIG. 1A depicts a Western blot analysis for total β-tubulin and β-tubulin isotypes I (β_{I}), II (β_{II}), III (β_{III}), and IV (β_{IV}) in the parental cell line (H460) and cell lines resistant to 30 nM each of Combretastatin A-4 (C30), paclitaxel (P30), and vinblastine (V30). Total cellular protein (10 ug) from each cell line was loaded on each lane of an SDS-PAGE gel, transferred to a PVDF membrane, and probed with monoclonal antibody raised against β-tubulin and tubulin isotypes I, II, III, and IV. Primary antibody was visualized with anti-mouse horseradish peroxidase secondary antibody and ECL chemiluminescence.

FIG. 1B is a graph depicting the relative intensity of each band of the Western blot. Each bar of the graph represents the normalized ratio of average pixel number for each tubulin isotype of a resistant cell line in relation to the parental H460 cell line.

### DETAILED DESCRIPTION OF THE INVENTION

Derived from the South African tree Combretum caffrum, combretastatins such as Combretastatin A-4 (CA-4) were initially identified in the 1980's as a potent inhibitors of tubulin polymerization. CA-4, and other combretastatins (e.g., CA-1) have been shown to bind a site at or near the colchicine binding site on tubulin with high affinity. In vitro studies clearly demonstrated that combretastatins are potent cytotoxic agents against a diverse spectrum of tumor cell types in culture. CA4P and CA1P, respective phosphate prodrugs of CA-4 and CA-1, were subsequently developed to combat problems with aqueous insolubility. Surprisingly, CA1P and CA4P have also been shown to cause a rapid and acute shutdown of the blood flow to tumor tissue that is separate and distinct from the anti-proliferative effects of the agents on tumor cells themselves. A number of studies have shown that combretastatins cause extensive shut-down of blood flow within the tumor microvasculature, leading to secondary tumor cell death (Dark et al., Cancer Res., 57: 1829-34, (1997); Chaplin et al., Anticancer Res., 19: 189-96, (1999); Hill et al., Anticancer Res., 22(3):1453-8 (2002); Holwell et al., Anticancer Res., 22(2A):707-11, (2002). Blood flow to normal tissues is generally far less affected by CA4P and CA1P than blood flow to tumors, although blood flow to some organs, such as spleen, skin, skeletal muscle and brain, can be inhibited (Tozer et al., Cancer Res., 59: 1626-34 (1999)).

In light of the novel, non-cytotoxic, mode of action of combretastatins, there is considerable interest in exploiting the novel “vascular targeting” of these agents for cancer treatment. Recently, single agent efficacy was reported for CA4P using a frequent dosing regimen. Another report suggested that large tumors can, in some cases, be more responsive to CA4P therapy than small tumors. However, many tumors harvested from animals treated with CA4P reveal central necrosis surrounded by a rim of viable cells (Dark et al., Cancer Res., 57: 1829-34, (1997); Chaplin et al., Anticancer Res., 19: 189-96, (1999)). This rim of surviving cells is most likely a consequence of the shared normal vessel circulation between the perimeter of tumors and neighboring normal tissue.

The vascular targeting and tumor cytotoxic activities of combretastatins are both the result of the tubulin biding properties of combretastatins. Combretastatins have been found to be among the most potent inhibitors of tubulin polymerization (Pettit et al., Experentia, 45: 209-11, (1989)).

The inventors have made the surprising discovery that tubulin isotypes are under-expressed in combretastatin-resistant cells. The inventors have established combretastatin resistant lung carcinoma lines and examined their β-tubulin isotype composition to determine if β-tubulin isotype patterns are related to cell resistance to combretastatins. Furthermore, the inventors have suggested that tubulin isotype distribution may be employed as a biomarker for improved therapeutic intervention in the treatment of solid tumors.

I. Definitions

As used herein, the terms “modulate”, “modulating” or “modulation” refer to changing the rate at which a particular process occurs, inhibiting a particular process, reversing a particular process, and/or preventing the initiation of a particular process. Accordingly, if the particular process is tumor growth or metastasis, the term “modulation” includes, without limitation, decreasing the rate at which tumor growth and/or metastasis occurs; inhibiting tumor growth and/or metastasis; reversing tumor growth and/or metastasis (including tumor shrinkage and/or eradication) and/or preventing tumor growth and/or metastasis.

As used herein, the term “prodrug” refers to a precursor form of the drug which is metabolically converted in vivo to produce the active drug. Thus, for example, combretastatin phosphate prodrug salts administered to an animal in accordance with the present invention undergo metabolic activation and regenerate combretastatin A-4 or combretastatin A-1 in vivo, e.g., following dissociation and exposure to endogenous non-specific phosphatases in the body.

As used herein, the terms “tumor”, “tumor growth” or “tumor tissue” can be used interchangeably, and refer to an abnormal growth of tissue resulting from uncontrolled progressive multiplication of cells and serving no physiological function. A solid tumor can be malignant, e.g., tending to metastasize and being life threatening, or benign. Examples of solid tumors that can be treated or prevented according to a method of the present invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, gastric cancer, pancreatic cancer, breast cancer, ovarian cancer, fallopian tube cancer, primary carcinoma of the peritoneum, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' tumor, cervical cancer, testicular tumor, lung carcinoma such as small cell lung carcinoma and non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Moreover, tumors comprising dysproliferative changes (such as metaplasias and dysplasias) can be treated or prevented with a pharmaceutical composition or method of the present invention in epithelial tissues such as those in the cervix, esophagus, and lung. Thus, the present invention provides for treatment of conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W. B. Saunders Co., Philadelphia, pp. 68 to 79). Hyperplasia is a form of controlled cell proliferation involving an increase in cell number in a tissue or organ, without significant alteration in structure or function. For example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. For a review of such disorders, see Fishman et al., 1985, Medicine, 2d Ed., J. B. Lippincott Co., Philadelphia.

Other examples of tumors that are benign and can be treated or prevented in accordance with a method of the present invention include arteriovenous (AV) malformations, particularly in intracranial sites and myoleomas.

As used herein, the term “biological sample” include, for example, a sample of blood, tissue (e.g., tumor tissue), serum, stool, urine, sputum, cerebrospinal fluid, cell supernatant from a cell lysate. In one exemplary embodiment, a tumor cell is employed as the biological sample in the methods of the invention. In another exemplary embodiment, an endothelial cell or endothelial tissue is employed as the biological sample in the methods of the invention.

II. Combretastatins

Therapeutic agents for use in the methods of the invention include combretastatins, prodrugs thereof, and their derivatives and analogs. Preferred combretastatins bind to tubulin and inhibit microtubule polymerization. Combretastatins are derived from tropical and subtropical shrubs and trees of the Combretaceae family, which represent a practically unexplored reservoir of new substances with potentially useful biological properties. Illustrative is the genus Combretum with 25 species (10% of the total) known in the primitive medical practices of Africa and India for uses as diverse as treating leprosy (See: Watt, J. M. et al, “The Medicinal and Poisonous Plants of Southern and Eastern Africa”, E. & S. Livingstone, Ltd., London, 1962, p. 194) (Combretum sp. root) and cancer (Combretum latifolium).

Combretastatins have been found to be antineoplastic substances. Numerous combretastatins have been isolated, structurally elucidated and synthesized. U.S. Pat. Nos. 5,409,953 and 5,569,786 describe the isolation and synthesis of Combretastatins designated as A-1, A-2, A-3, B-1, B-2, B-3 and B-4. The disclosures of these patents are incorporated by reference herein in their entirety. A related Combretastatin, designated Combretastatin A4, was described in U.S. Pat. No. 4,996,237 to Pettit et al., which is incorporated by reference herein in its entirety.

Many combretastatins are characterized by poor aqueous solubility. This characteristic interferes with the formulation of pharmaceutical preparations of these compounds. Thus, prodrug forms of combretastatins have been introduced which compensate for the generally poor solubility of these agents and include enzymatically labile moieties which allow for metabolic back to water insoluble parent drug in vivo. Suitable prodrugs include, inter alia, the phosphate prodrugs described in U.S. Pat. No. 5,561,122 and PCT publications WO 02/22626 and WO 99/35150, the disclosures of which are incorporated herein.

An exemplary combretastatin prodrug is the diphosphate prodrug of CA1, Combretastatin A-1 diphosphate (“CA1P” , also known as OXI4503), which has the following structure:

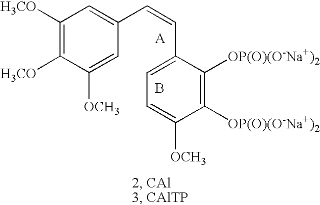

The present invention also contemplates the use of synthetic analogs of combretastatins, or prodrugs thereof. Derivatives or analogs of combretastatins are described in Singh et al., J. Org. Chem., 1989; Cushman et al, J. Med. Chem., 1991; Getahun et al, J. Med. Chem., 1992; Andres et al, Bioorg. Med. Chem. Lett., 1993; Mannila, et al., Liebigs. Ann. Chem., 1993; Shirai et al., Bioorg. Med. Chem. Lett., 1994; Medarde et al., Bioorg. Med. Chem. Lett., 1995; Wood et al, Br. J. Cancer, 1995; Bedford et al., Bioorg. Med. Chem. Lett., 1996; Dorr et al., Invest. New Drugs, 1996; Jonnalagadda et al., Bioorg. Med. Chem. Lett., 1996; Shirai et al., Heterocycles, 1997; Aleksandrzak, et al., Anticancer Drugs, 1998; Chen et al., Biochem. Pharmacol., 1998; Ducki et al., Bioorg. Med. Chem. Lett., 1998; Hatanaka et al., Bioorg. Med. Chem. Lett., 1998; Medarde et al, Eur. J. Med. Chem., 1998; Medina et al., Bioorg. Med. Chem. Lett., 1998; Ohsumi et al., Bioorg. Med. Chem. Lett., 1998; Ohsumi et al., J. Med. Chem., 1998; Pettit, et al., J. Med. Chem., 1998; Shirai et al., Bioorg. Med. Chem. Lett., 1998; Banwell et al., Aust. J. Chem., 1999; Medarde et al., Bioorg. Med. Chem. Lett., 1999; Shan et al., PNAS, 1999; Combeau et al., Mol. Pharmacol., 2000; Pettit et al., J. Med. Chem., 2000; Pinney et al., Bioorg. Med. Chem. Lett., 2000; Flynn et al., Bioorg. Med. Chem. Lett., 2001; Gwaltney et al., Bioorg. Med. Chem. Lett., 2001; Lawrence et al., 2001; Nguyen-Hai et al., Bioorg. Med. Chem. Lett., 2001; Xia et al., J. Med. Chem., 2001; Tahir et al., Cancer Res., 2001; Wu-Wong et al., Cancer Res., 2001; Janik et al, Biooorg. Med. Chem. Lett., 2002; Kim et al., Bioorg Med Chem Lett., 2002; Li et al., Biooorg. Med. Chem. Lett., 2002; Nam et al., Bioorg. Med. Chem. Lett., 2002; Wang et al, J. Med. Chem. 2002; Hsieh et al., Biooorg. Med. Chem. Lett., 2003; Hadimani et al., Bioorg. Med. Chem. Lett., 2003; Mu et al., J. Med. Chem, 2003; Nam et al., Curr. Med. Chem., 2003; Pettit et al, J. Med. Chem., 2003; WO2005/007635, WO2005/007603, WO2004/078126, WO 03/040077, WO 03/035008, WO 02/50007, WO 02/14329; WO 01/12579, WO 01/09103, WO 01/81288, WO 01/84929, WO 00/48590, WO 00/73264, WO 00/06556, WO 00/35865, WO 99/34788, WO 99/48495, WO 92/16486, U.S. Pat. Nos. 6,794,384; 6,787,672, 6,777,578, 6,723,858, 6,720,323, 6,433,012, 6,423,753, 6,201,001, 6,150,407, 6,169,104, 5,731,353, 5,674,906, 5,430,062, 5,525,632, 4,996,237 and 4,940,726, which are incorporated herein by reference in their entirety.

In one embodiment, combretastatin analogs include compositions of the following formula I: 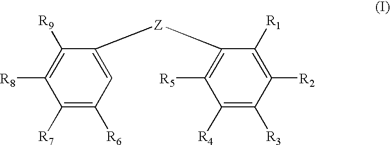
wherein
a. Z is a chemical bridge comprising 1-4 contiguous atoms from a chemical group selected from the group consisting of an alkene (—CR₉═CR₁₀—), an alkane (—CR₉—CR₁₁R₁₂), an alkyne, an amide(—NR₉—CO—), an amine(—NH—, —NR₈—, or —CR₉—N—), a carbonyl (—CO—), an ether (—C R₈—O—), a sulfonamide (—NR8—SO2—), a sulfonate (—O—SO₂—), an aryl (including optionally substituted aromatic heterocycles such as furans or benzo[b]furans, furanones, thiophenes or benzo[b]thiophenes, dioxazoles, imidazoles, indoles, indanes, indenes, lactams, naphthalenes, oxazoles, oxazolines, oxazolones, oxadiazolines, pyrazoles, thiazoles, thiophenes, triazoles, or tetrazoles), an oxo (—O— or —OR₈—), a thio (—S—), a cycloalkyls, a propanone (—(C═O)—CR₈═CR₉—), a sulfonamide (—NR₈—(S═O)₂—), or a sulfonate (—O—(S═O)₂—), wherein R₈, R₉, R₁₀, or R₁₁ are alternatively H, halo (e.g., fluoro, bromo, etc.), alkyl, amino, amido, cyano, hydroxyl, or carboxy.
b. at least one of R₁, R₂, R₃, R₄, R₅, R₆, R₇, R₈, and R₉ are optionally
   i) a C₁, C₂, C₃, C₄ or C₅ (preferably C₁) branched or straight-chain lower alkoxy, cycloalkoxy, heterocycloalkoxy, aryloxy, lower alkanoyloxy;
   ii) a halogen or trihaloalkyl;
   iii) a C₁, C₂, C₃, C₄ or C₅ (preferably Cl) branched or straight chain lower alkyl, allyl, allyloxy, vinyl, vinyloxy;
   iv) OH, or a C₁, C₂, C₃, C₄ or C₅ (preferably C₁) primary, secondary, or tertiary alcohol;
   v) NH_{2,} amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, aralkanoylamido; or
   vi) oxo, lower alkanoyl, thiol, sulfonyl, sulfonamide, nitro, nitrosyl, cyano, carboxy, carbamyl, aryl, heterocyclo; and
c. the remaining R₃, R₄, R₅, R₆, R₇, R₈, and R₉ are hydrogen.

In a more preferred embodiment, combretastatin analogs include compositions of the following formula Ia. 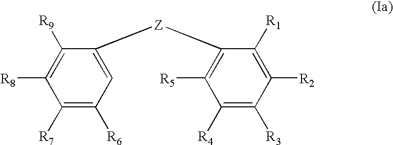
wherein
a. Z is a is an ethylene group (—CH═CH—);
b. At least one of R₁ and R₂ is OH;
c. R_{6,} R₇, and R₈ are methoxy;
d. at least one of R₁, R₂, R₃, R₄, R₅,R₆, R₇, R₈, and R₉ is optionally
   i) a C₁, C₂, C₃, C₄ or C₅ (preferably C₁) branched or straight-chain lower alkoxy, cycloalkoxy, heterocycloalkoxy, aryloxy, lower alkanoyloxy;
   ii) a halogen or trihaloalkyl;
   iii) a C₁, C₂, C₃, C₄ or C₅ (preferably C₁) branched or straight chain lower alkyl, allyl, allyloxy, vinyl, vinyloxy;
   iv) OH, or a C₁, C₂, C₃, C₄ or C₅ (preferably C₁) primary, secondary, or tertiary alcohol;
   v) NH₂, amino, lower alkylamino, arylamino, aralkylamino, cycloalkylamino, heterocycloamino, aro ylamino, aralkanoylamino, amido, lower alkylamido, arylamido, aralkylamido, cycloalkylamido, heterocycloamido, aroylamido, aralkanoylamido; or
   vi) oxo, lower alkanoyl, thiol, sulfonyl, sulfonamide, nitro, nitrosyl, cyano, carboxy, carbamyl, aryl, heterocyclo; and
e. the remaining R₃, R₄, R₅, R₆, R₇, R₈, and R₉ are hydrogen.

The compounds contemplated for use in the methods of the invention include, without limitation, all pharmaceutically-acceptable salts of those compounds. As used herein, the term “pharmaceutically acceptable salt” refers to a substantially non-toxic salt of a compound of the invention with the desired pharmacokinetic properties, palatability, and solubility, for use in medicine. When the compounds of the present invention contain a basic group, salts encompassed within the term “pharmaceutically acceptable salts” refer to non-toxic salts which are generally prepared by reacting the free base with a suitable organic or inorganic acid (e.g., an acid-addition salt). Representative salts include the following: amino acids, acetates, ascorbates, benzoates, bicarbonates, bisulfates, bitartrate, borate, bromide, calcium, camsylates, carbonates, chlorides, clavulanates, citrates, edetates, edisylates, estolates, esylates, fumarates, glycolates, gluceptates, gluconates, glutamates, glycollylarsanilates, hydrochlorides, hexylresorcinates, hydrabamines, hydrobromides, hydrochlorides, iodides, isothionates, hydroxynaphthoates, hydrobromides, lactates, lactobionates, laurates, malates, maleates, mandelates, mesylates, methylbromides, methylnitrates, methylsulfates, mucates, napsylates, nitrates, oleates, oxalates, pamoates (embonates), palmitates, pantothenates, phosphates, polygalacturonate, sulphates, sulphonates, stearates, salicylates, succinates, suceinates, tannates, tartrates, teoclates, tosylates, triethiodides and valerates. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic bases, e.g., dicyclohexylamine, tributylamine, pyridine, triethylamine, and as others disclosed in PCT International Application Nos. WO02/22626 or WO00/48606. The salts of the present invention can be synthesized by conventional chemical methods. Generally, the salts are prepared by reacting the free base or acid with stoichiometric amounts or with an excess of the desired salt-forming inorganic or organic acid or base, in a suitable solvent or solvent combination.

A. Assessment of Combretastatin Resistance

Many of the most common carcinomas, including breast and ovarian cancer, are initially relatively sensitive to a wide variety anti-cancer agents. However, acquired drug resistance phenotype typically occurs after months or years of exposure to chemotherapy. Determining the molecular basis of drug resistance may offer opportunities for improved diagnostic and therapeutic strategies. Therefore, the present invention contemplates the assessment of drug resistance in a patient suffering from a cancer or tumor comprising evaluating the tubulin isotype profile of the patient. In one embodiment, the total expression level of tubulin in a biological sample of the patient is measured. In another embodiment, the relative levels of each tubulin isotype in the patient is measured.

i) Tubulin

Microtubules, cellular organelles present in all eukaryotic cells, are required for healthy, normal cellular activities. They are an essential component of the mitotic spindle needed for cell division, and are required for maintaining cell shape and other cellular activities such as motility, anchorage, transport between cellular organelles, extracellular secretory processes (Dustin, P. (1980) Sci. Am., 243: 66-76), as well as modulating the interactions of growth factors with cell surface receptors, and intracellular signal transduction. Furthermore, microtubules play a critical regulatory role in cell replication as both the c-mos oncogene and CDC-2-kinase, which regulate entry into mitosis, bind to and phosphorylate tubulin (Verde, F. et al. (1990) Nature, 343:233-238), and both the product of the tumor suppressor gene, p53, and the T-antigen of SV-40 bind tubulin in a ternary complex (Maxwell, S. A. et al. (1991) Cell Growth Differen., 2:115-127).

Microtubules are not static, but are in dynamic equilibrium with their soluble protein tubulin subunits. Microtubules are heterodimeric proteins which are in turn comprised of α- and β-tubulin subunits. Assembly under physiologic conditions requires guanosine triphosphate (GTP) and certain microtubule associated and organizing proteins as cofactors; on the other hand, high calcium and cold temperature cause depolymerization. Interference with this normal equilibrium between the microtubule and its tubulin subunits would therefore be expected to disrupt cell division and motility, as well as other activities dependent on microtubules. Accordingly many anti-cancer agents, including combretastatins, have been shown to have these tubulin modulating activity.

Photoaffinity labeling and other binding site elucidation techniques have identified three key binding sites on β-tubulin for tubulin binding agents: 1) the colchicine site at its interface with the α-tubulin monomer (Bai et al, 1996; Uppuluri 1993) (Floyd et al, Biochemistry, 1989; Staretz et al, J. Org. Chem., 1993; Williams et al, J. Biol. Chem., 1985; Wolff et al, Proc. Natl. Acad. Sci. U.S.A., 1991), 2) the vinca alkaloid site at residues 175-213 (Rai and Wolff, 1996; Safa et al, Biochemistry, 1987), and 3) a taxoid site near the M loop of β-tubulin to which taxol binds in polymerized microtubules (Rao et al, J. Natl. Cancer Inst., 1992; Lin et al, Biochemistry, 1989; Sawada et al, Bioconjugate Chem, 1993; Sawada et al, Biochem. Biophys. Res. Commun., 1991; Sawada et al, Biochem. Pharmacol., 1993).

The two 50 kDa proteins of tubulin, α- and β-tubulin, exist as multiple sequence variants or isotypes. Human β-tubulin exists as six distinct isotypes designated as classes I, II, III, IVₐ, IV_{b}, VI (Sullivan, 1988; Sullivan and Cleveland, 1986; Cowen and Dudley, 1983). These isotypes represent a highly homologous family of proteins that are well conserved between species and are grouped into different classes by the extreme unique carboxyl-terminal region (Sullivan, 1988).

B. Assessment of Tubulin Isotype Profiles

Tubulin isotype expression can be determined by a variety of methods. In one embodiment, the level of an mRNA encoding a tubulin can be measured using methods known to those skilled in the art, e.g., Northern analysis. In another embodiment, the level of a tubulin isotype protein can be determined using well-known methods, e.g., Western blotting.

Alternatively, patients with acquired or intrinsic combretastatin resistance can be identified by obtaining tumor tissue sample and conducting sequence or expression analysis of genes associated with tubulin isotypes using techniques that are well-known in the art. The present invention also provides a diagnostic assay for identifying whether a cell is cancerous.

The method includes: (a) providing a cell which is suspected of being cancerous and (b) determining the tubulin isotype expression profile in the cell. Decreased expression of tubulin isotype III or IV is an indication that the cell is resistant to a combretastatin. Conversely, decreased expression of tubulin isotype III or IV is an indication that the cell is susceptible to a combretastatin. Tubulin isotype expression can be detected through measurement of tubulin isotype RNA level in the cell, or through measurement of tubulin isotype polypeptide level in the cell.

In one embodiment, assessment of drug resistance is performed in a patient suffering from a cancer or tumor which is refractive to treatment with anticancer therapy comprising a combretastatin and optionally one or more additional anticancer agents. Refractive tumors or cancers can be identified as those tumors from patients who have developed resistance to combretastatin during the course of treatment with a combretastatin. Accordingly, an analysis of tubulin isoform expression in such a patient may be performed in order to determine if the patient has acquired resistance to the combretastatin or some other agent that is co-administered with the combretastatin. An enhanced expression of tubulin isoform is evidence of acquired combretastatin resistance. Accordingly, the patient may be administered a different anticancer therapy so that prognosis may be increased.

In another aspect, an analysis of tubulin expression may be performed in a patient who has not yet received anti-cancer therapy. An analysis of tubulin expression may be performed to determine if the patient is likely to respond to treatment with a combretastatin. An increase in tubulin isotype expression in such a patient is indicative of intrinsic susceptibility to the combretastatin. Accordingly, such a patient may be selected for treatment with a combretastatin. Conversely, an decrease in tubulin isotype expression in the patient is indicative of intrinsic resistance to the combretastatin. Such a patient may be selected for treatment with an anticancer agent that is not a combretastatin.

Tubulin isotype levels may be determined by any acceptable method that is known in the art. In one embodiment, tubulin isotype levels may be measured directly by measuring (e.g., counting) the tubulin isotype as a percentage of total protein in a biological sample obtained from a patient treated with an anti-cancer agent. In another embodiment, the amount of tubulin isotype III or IV as a percentage of all total tubulin can be measured. Methods for measuring the tubulin isotype proteins include Western blotting and ELISA. The value for the amount of tubulin III or IV isotype in a cells may be an absolute or relative value (e.g., a tubulin III:total tubulin ratio). In another embodiment, tubulin levels are measured indirectly by measuring the amount of tubulin RNA encoding the tubulin isotype. Methods for measuring tubulin isotype RNA include RT-PCR, in situ RNA hybridization, and Northern blotting.

C. Diagnostic Kits for Combretastatin Resistance

In one aspect, the invention provides diagnostic kits for evaluating tubulin isotype profiles in a biological sample obtained from a patient, said kit comprising a tubulin expression probe. Suitable probes include, for example, cDNA, oligonucleotides, riboprobes, and antibodies. The type of probe used will generally be dictated by the particular situation, such as riboprobes for in situ RNA hybridization, cDNA for Northern blotting, DNA oligonucleotide for RT-PCT, and antibodies for Western Blotting or ELISA. The most preferred probes are those directed to nucleotide or polypeptide regions that are unique to the tubulin III or IV isotype. The form of labeling of the probes may be any that is appropriate, such as the use of radioisotopes. Labeling with radioisotopes may be achieved, whether the probe is synthesized is chemically or biologically, by the use of suitably labeled bases. Other forms of labeling may include enzyme or antibody labeling such as is characteristic of ELISA.

D. Methods for Selecting Patients and Prognosticating Treatment

In another aspect, the invention provides methods for selecting patients for treatment with the anti-cancer agents disclosed herein, in particular a combretastatin compound, as well as methods for prognosticating the response of the patient to the treatment, and methods for monitoring the course of treatment with the anticancer agent.

The methods include determining the level of a biomarker in a biological sample derived from a patient previously treated with the anti-cancer agent. The methods of the invention employ tubulin isotype levels, in particular tubulin isotype III or UV levels, as a biomarkers. Tubulin isotypes

The inventors have discovered that tubulin isotype levels (e.g., tubulin isotypes III or IV) are decreased in combretastatin-resistant cells and are correlated with tumor response, such a biomarker may be employed as a surrogate marker of clinical efficacy.

A method for selecting a patient for further treatment with an anticancer agent (e.g., a combretastatin) may be based on the level of tubulin isotype biomarker observed in a biological sample obtained from the patient. The method comprises treating the patient with a first dose of anti-cancer agent, obtaining a biological sample from the patient, and measuring the level of tubulin isotypes in the biological sample, and selecting the patient for treatment based at least in part on the level obtained. The patient may be selected for continued treatment with the anticancer agent if decreased tubulin isotype levels are observed following initial treatment with the anticancer agent. Alternatively, if tubulin isotype levels decrease or remain constant following treatment, the patient may be advised to discontinue treatment with the anticancer agent.

In another aspect, tubulin isotype levels may be used to monitor the progression of cancer in the patient following treatment with an anti-cancer agent (e.g., a combretastatin). The methods include determining the tubulin isotype level in the patient at a first time following treatment with the anti-cancer agent, determining the tubulin isotype level in the patient at a subsequent time following a treatment with the anticancer agent. For example, the first measurement may be performed at a time just following a first dose of anti-cancer agent, while a second measurement may be performed at a second time that follows the first dose or a second or subsequent dose of anti-cancer agent. Tubulin isotype levels obtained at said first time and second times may then be compared. Decreased levels of tubulin isotypes at the second time relative to the first time may be used to support a diagnosis that the tumor has progressed or relapsed (i.e., continued growth of the tumor). Decreased levels of tubulin isotypes at the second time relative to the first time may be used to support a diagnosis that the tumor has regressed (i.e., tumor shrinkage).

In another aspect, the invention provides a method of assessing, predicting or prognosticating the likelihood of a patient's response (e.g., tumor regression or remission) to treatment with an anti-cancer agent, and in particular a combretastatin. Efficacy of anti-cancer agents can be predicted and the probable clinical course of a patient suffering from cancer can be determined by measuring tubulin isotype levels in a biological sample obtained from the patient. For example, an increase in tubulin isotype levels correlates with the increased likelihood of a tumor response. The presence of reduced tubulin isotype levels in the biological sample of the patient is indicative of a response of the tumor to treatment with the anti-cancer agent (e.g., a combretastatin). Conversely, higher levels than certain baselines can also be used to indicate the lack of response of the tumor to treatment. For example, an elevation of tubulin isotype III or IV levels is determined by comparing the post-treatment isotype III or IV levels with a baseline level (e.g., the same or different patient than prior to treatment with the anticancer agent). Such a patient is predicted to have a favorable prognosis.

Increases or decreases in relative or absolute tubulin isotype levels of more than 1.0% from baseline may be used to make any of the determinations described above. Preferably, the increase or decrease in tubulin isotype levels, is greater than 2.0%, 3.0%, 4.0%, 5.0%, 7.0%, 10%, 15%, 20%, 25%, 30%, 40%, 50%, or more. While the exact baseline level is somewhat arbitrary (as the numerical cut off value may be shifted upward or downward with an attendant loss of accuracy in the prognostic utility of the test), it is well within the skill of one of ordinary skill in the art to determine the appropriate baseline level, by either using the experimental methods disclosed herein, for example, establishing tubulin isotype levels in patients that have not been administered the anti-cancer agent that is evaluated. Further, as will be appreciated by those of ordinary skill in the art, the evaluation of the treatment may also be based upon an evaluation of the symptoms or clinical end-points of the associated disease.

The comparison of a subject's tubulin isotype levels employs measurements obtained from biological samples collected from the subject at different sample times. A first biological sample, if necessary, may be obtained at any time prior to treatment with an anti-cancer agent. A second biological sample is preferably obtained within 24 hours of treatment with the anticancer agent (e.g., a combretastatin). In more preferred embodiment, a second biological sample is obtained less than 6 hours following the administration of an anticancer agent (e.g., a combretastatin). The preferred time to obtain the second biological sample from the subject is at 4 hours following the administration of the anticancer agent.

Biological samples which can be screened for granulocyte levels are samples containing tubulin isotypes, preferably tumor cells or vascular endothelial cells. Examples include, but are not limited to, tumor biopsy samples and blood or serum samples obtained from the patient. In a preferred embodiment, the biological sample is obtained from the blood of the patient. The following examples are provided to illustrate embodiments of the invention. They are not intended to limit the invention in any way.

### EXAMPLES

The following protocols are provided to facilitate the practice of Example I.

Drugs

Combretastatin A-4 (CA-4) was obtained from PHARM-ECO (Devens, Mass., USA). Paclitaxel, vinblastine and verapamil were purchased from ICN Biomedicals (Aurora, Ohio, USA), and cisplatin from Sigma (St. Louis, Mo., USA). Stock solutions were prepared in dimethyl sulfoxide (DMSO) and stored at −20° C. The working solutions were diluted in culture medium to the desired concentration before use; the highest concentration of DMSO was 0.1% (v/v).

Cell Culture

A panel of cells lines obtained from American Type Culture Collection (Rockville, Md., USA) was initially assayed against CA-4 to screen for drug sensitivity. The human non-small cell lung carcinoma (NSCLC), NCI-H460, was selected for resistance based on significant cytotoxicity. H460 cells were grown and maintained in RPMI-1640 supplemented with penicillin-streptomycin, Fungizone® and 10% heat inactivated fetal bovine serum (Invitrogen, Carlsbad, Calif., USA) and maintained at 37° C. humidified 5% CO₂ atmosphere. H460 parental cell line was selected for resistance to CA-4, paclitaxel or vinblastine by incubation with stepwise increments of each drug, beginning at 2 nM. Cells were treated with the drugs for 24 h, once a week, and every 2-3 weeks, drug dose was increased by 2 nM increments until 30 nM drug resistance was reached. To select non-MDR resistant cell lines, cells were evolved in the presence of verapamil, an inhibitor of P-glycoprotein (P-gp) drug efflux pump that produces a MDR phenotype. Resistance was maintained by acute 24 h exposure to the corresponding drug and verapamil once a month. All cells used for the experiments were grown logarithmically in drug-free medium at least a week before experiments.

In vitro Cytotoxicity Assay

Cytoxicity assays was assessed using a modified MTT assay (Mosmann, 1983). MTT (3-(4,5-dimethylthiazol-2-yl)-2,5-diphenyl tetrazolium bromide, MTT) was purchased from Sigma (St. Louis, Mo., USA). For each drug treatment, triplicate wells were seeded (5.0×10³/well) and conditioned overnight. Drugs were prepared by serial dilution with RPMI media and added to the corresponding wells. After 48 h, the MTT assay was performed. Percentage survival was calculated as the OD₅₆₀ of the treated sample divided by the OD₅₆₀ of the untreated control cell line. Cell lines were tested with cisplatin to test for non-MDR phenotype.

Protein Extraction and Western Blotting

Cell lines were grown to confluence, pelleted and washed with cold PBS. Cells were lysed with 500 μl of ice-cold buffer (150 mM NaCl, 1% NP-40, 0.1% SDS, 50 mM Tris, pH 7.5, 2 mM PMSF, 5 mM ε-aminocaproic acid, 1 mM benzamide, 10 μg/ml apoprotein, 100 μg/ml soybean trypsin inhibitor, 1 μg/μl leupeptin and 500 μl β-mercaptoethanol) for 30 minutes at 4° C. Samples were centrifuged at 10,000×g for 10 minutes at 4° C. The supernatants containing total cellular protein were quantified using the BioRad protein assay reagent (BioRad, Hercules, Calif., USA).

Equal amounts of total protein (10 μg) were loaded onto 10% SDS-PAGE gel and stained with Coomassie blue to confirm equal loading per cell line. Gels and blotters were equilibrated in modified Towbin transfer buffer (25 mM Tris, 192 mM glycine, 20% methanol) for 15 min prior to transfer. PVDF membrane was pre-wet with methanol for 15 sec and then with transfer buffer for 15 min. Protein transfer was performed at 10 V for 20 min per 1 mini-gel with BioRad Trans-Blot® Semi-Dry Transfer Cell. The gel was stained to confirm complete transfer of protein. Equal loading and efficient transfer of proteins were confirmed by staining the membrane with Ponceau S red before immunodetection. The membrane was blocked for 1 h at RT with 5% dried nonfat milk in TBS-Tween buffer (20 mM Tris-Cl, pH 7.5, 500 mM NaCl, 0.05% (v/v) Tween 20) and then incubated for 1 h at RT with monoclonal mouse antibodies against P-gp, β-, β_{I}, β_{II}-, β_{III}-, or β_{IV}-tubulin (1:500, Sigma, St. Louis, Mo., USA). The membrane was incubated for 1 h with horseradish peroxidase linked rabbit anti-mouse secondary antibody (1:10,000, Sigma, St. Louis, Mo., USA), washed, and developed with enhanced chemiluminescence (ECL) reagent (Amersham, Arlington Heights, Ill., USA). Protein expression was detected using a Fluro S Max Image (BioRad, Hercules, Calif., USA) and quantified with Quantity One densitometry software (BioRad, Hercules, Calif., USA). Results were reported as a normalized ratio of average volume total pixel number of the resistant cell to the parental H460 cell. Western blots were obtained from at least three independent experiments.

### Example I

### Analysis of Tubulin Expression in CA-4 Resistant Cell Lines

To select a moderately sensitive cell line for drug-resistance, a panel of susceptible tumor cell lines was screened for CA-4 cytotoxicity. Table 1 depicts the average in vitro cytotoxicity (IC50) values for three independent experiments with each experiment run in triplicate.

TABLE 1
In vitro cytotoxicity assays of susceptible tumor cell lines

| Cancer Type | Cell Line | IC50 (nM) |
|---|---|---|
| Acute Lymphoblastic | CEM | 0.25 ± 0.03 |
| Leukemia | | |
| Acute Lymphoblastic | MOLT4 | 4.1 ± 0.61 |
| Leukemia | | |
| Colorectal | HT29 | 5.51 × 10⁴ ± 0.88 |
| Adenocarcinoma | | |
| Mammary Gland | MCF7 | 1.65 × 10⁵ ± 0.31 |
| Adenocarcinoma | | |
| Non-Small Cell Lung | H460 | 7.3 ± 1.37 |
| Carcinoma | | |
| Pancreatic | BXPC3 | 5.7 × 10⁴ ± 0.00 |
| Adenocarcinoma | | |
| Prostate Carcinoma | DU145 | 7.7 ± 1.96 |

Leukemia cell lines CEM and MOLT-4 showed high sensitivity with IC₅₀ values of 0.25 nM and 4.1 nM, respectively. Prostate (DU-145) and NSCLC-H460 carcinomas displayed high sensitivity with IC₅₀ values of 7.7 nM and 7.3 nM. Pancreatic (BXPC-3), mammary (MCF-7), and colorectal (HT29) cancer cell lines had much lower sensitivity to CA-4 with IC₅₀ values from 5.5×10⁴ nM to 1.65×10⁵ nM. From this data, H460 cell line was selected for drug-resistance against CA-4 as it showed significant cytotoxicity and a similar IC₅₀ value to paclitaxel and vinblastine.

i) Resistance and Cross-Resistance of Cell Lines

Using step-wise selection, drug resistance to CA-4, paclitaxel or vinblastine was established in the NSCLC-H460 cell line in the presence of verapamil, a P-gp drug efflux pump inhibitor. C30, P30, and V30 cell lines represent H460 parental cells selected at 30 nM CA-4, paclitaxel, and vinblastine, respectively. Resistance was non-MDR-1 mediated as levels of P-gp protein were undetectable by western analysis (data not shown). As shown in Table 2, C30 cells were threefold resistant to CA-4, and cross-resistance studies showed that the cells were tenfold resistant to paclitaxel and negligibly resistant to vinblastine. P30 cells were tenfold resistant to paclitaxel and threefold cross-resistance to vinblastine; cells showed negligible resistance to CA-4. V30 cells were fivefold resistant to vinblastine, eleven fold resistant to paclitaxel and negligibly resistant to CA-4. Cisplatin, an anti-cancer agent with a non-tubulin-binding mode of action was assayed against the cell lines to test for MDR phenotype. Compared to the parental cell line, C30, P30 and V30 cytotoxicity to cisplatin was not altered (see Table 2).

TABLE 2
In vitro cytotoxicity assay of resistant tumor cell lines

| | H460 | C30 | | P30 | | V30 | |
|---|---|---|---|---|---|---|---|
| Tubulin | wild-type | | Resistance | | Resistance | | Resistance |
| Binding Agent | IC50 (nM) | IC50 (nM) | Factor | IC50 (nM) | Factor | IC50 (nM) | Factor |
| CA-4 | 7.0 ± 0.6 | 19.0 ± 1.7 | 2.7 ± 0.4 | 8.5 ± 0.2 | 1.2 ± 0.1 | 9.2 ± 0.6 | 1.3 ± 0.1 |
| Paclitaxel | 8.1 ± 0.4 | 80.0 ± 2.0 | 9.9 ± 0.6 | 77 ± 4.9 | 9.6 ± 1.0 | 87 ± 3.6 | 10.8 ± 1.0 |
| Vinblastine | 5.9 ± 0.1 | 6.0 ± 0.3 | 1.0 ± 0.0 | 15.7 ± 04.2 | 2.7 ± 0.7 | 26.3 ± 2.1 | 4.5 ± 0.4 |
| Cisplatin | 20 ± 2.6 | 16.7 ± 3.2 | 0.8 ± 0.1 | 25.3 ± 3.1 | 1.3 ± 0.0 | 21.7 ± 0.6 | 1.1 ± 0.2 |

β-Tubulin Isotype Analysis

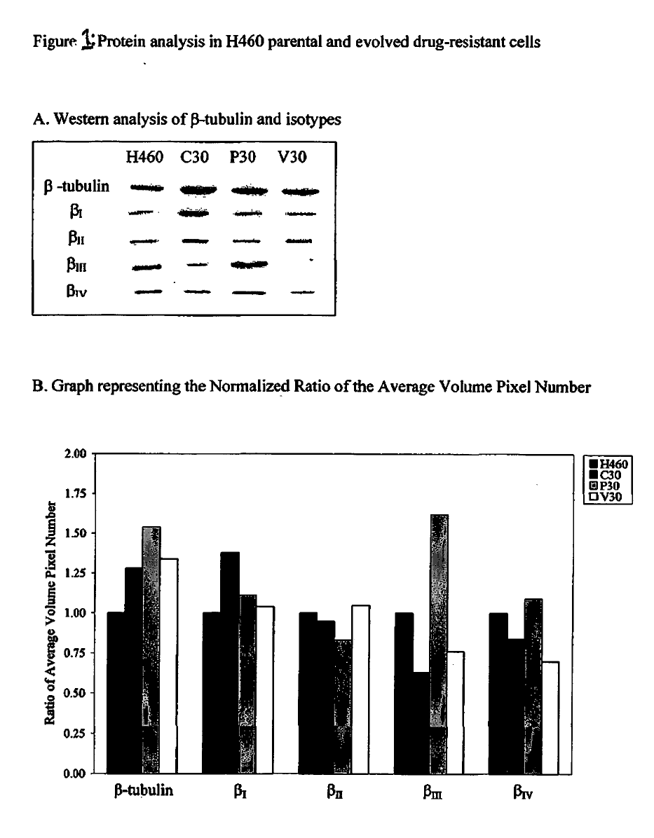

Beta-tubulin and isotypes I, II, III, and IV were analyzed by western blotting and compared to parental cell line (see FIG. 1). Total β-tubulin was increased in all resistant cell lines with P30 having the highest increase at 1.5-fold. C30 cells showed an increase in β_{I}-tubulin, with a 1.4-fold increase. P30 cells showed a 1.2-fold decrease of β_{II}-tubulin. Class III tubulin was altered in resistant cell lines, with P30 cells showing an increase of 1.6-fold and C30 and V30 showing a reduction by 1.6- and 1.3-fold, respectively. C30 and V30 also demonstrated a decrease in βIV-tubulin with reductions of 1.2 and 1.4-fold, respectively.

Conclusion

The results of this study demonstrate that resistance to CA-4, paclitaxel, and vinblastine, three agents binding distinct tubulin sites, confers altered β-tubulin isotype expression levels in NSCLC-H460. Agents that bind to the colchicine and Vinca alkaloid sites inhibit microtubule polymerization and promote depolymerization whereas the taxoids stabilize polymerized microtubules and maintain the polymerized state. To our knowledge, this is the first report of the establishment of CA-4 resistance and characterization of β-tubulin isotype alterations; in addition, this study demonstrates altered β-tubulin isotypes in any cell line resistant to an agent that binds at the colchicine site of tubulin and suggests that resistance to tubulin binding agents with similar modes of action, i.e., microtubule depolymerizers or polymerizer, confer similar alterations in β-tubulin isotypes.

After five months of similar treatment, the level of resistance to the polymerizing stabilizer, paclitaxel, evolved faster than the depolymerizing agents, CA-4 and vinblastine. The P30 cells had a tenfold resistance whereas the C30 and V30 cells were three and fivefold resistant at 30 nM of drug. Interestingly, at 30 nM of CA-4 resistance, cells demonstrated an IC₅₀ of 19 nM indicating the possibility of resistance instability. Previous studies with other drugs have shown IC₅₀ values of resistant cell lines at the maximum treated dose, but not at a lower concentration (Ranganathan et al, 1996; Han et al, 2000; Chu et al, 2000). This trait is a unique characterization of the CA-4 resistant cell line and is not true for previous resistant studies found in the literature.

Western analysis results suggest that multidrug resistance did not play a role in establishing resistance as an increased expression of the drug efflux pump P-gp was not detected in the resistant cell lines. Because an increase of isotypes III and IV is demonstrated in P30 cells, a decreased expression of the isotypes should increase sensitivity to paclitaxel. Interestingly, cross-resistance studies of C30 and V30 cells with paclitaxel (Table 2) did not demonstrate this expected increase of sensitivity. In fact, the cells showed a tenfold resistance to paclitaxel. A previous drug-resistant cell line study in which the multidrug resistant phenotype was not present also revealed a similar cross-resistance to tubulin-binding agents (Ohta et al, 1993). We believe that this cross-resistance is due to the instability of microtubule dynamics; however, the possibility of any other drug efflux system and drug-resistance mechanisms has not been evaluated.

1. A method for determining the prognosis of a patient suffering from cancer, wherein said patient has been administered a combretastatin, the method comprising:
(a) obtaining a biological sample from the patient;
(b) determining a tubulin isotype level of the biological sample;
(c) comparing the tubulin isotype level with a baseline level; and
(d) correlating the tubulin isotype level with an indication of unfavorable prognosis if the tubulin isotype level is less than the baseline level or correlating the neutrophil level with an indication of favorable prognosis if the tubulin isotype level is greater than the baseline,
thereby determining the prognosis of the patient.

2. The method of claim 1, wherein said combretastatin is combretastatin A-4.

3. The method of claim 1, wherein said tubulin isotype level is selected from the group consisting of tubulin isotype III and tubulin isotype IV.

4. The method of claim 1, wherein said biological sample is obtained less than 24 hours after treatment with the combretastatin.

5. The method of claim 1, wherein said biological sample is obtained less than 6 hours after treatment with the combretastatin.

6. A method for selecting a patient for further treatment with a combretastatin, the method comprising:
(a) determining a tubulin isotype level in a first biological sample from the patient;
(b) administering the combretastatin to the patient;
(c) determining a second tubulin isotype level from a second biological sample obtained from the patient;
(d) comparing the first and second tubulin isotype levels; and
(e) selecting the patient for further treatment if a decrease in tubulin isotype level is observed.

7. The method of claim 6, wherein said combretastatin is combretastatin A-4.

8. The method of claim 6, wherein said tubulin isotype level is selected from the group consisting of tubulin isotype III and tubulin isotype IV.

9. The method of claim 6, wherein said biological sample is obtained less than 24 hours after treatment with the combretastatin.

10. The method of claim 6, wherein said biological sample is obtained less than 6 hours after treatment with the combretastatin.

11. A method for monitoring the progression of a tumor in patient, the method comprising:
(a) determining a tubulin isotype level in a first biological sample from the patient;
(b) administering a combretastatin to the patient;
(c) determining a second tubulin isotype level from a second biological sample obtained from the patient; and
(d) comparing the first and second tubulin isotype levels,
thereby monitoring the progression of the tumor in the patient.

12. The method of claim 11, wherein said combretastatin is combretastatin A-4.

13. The method of claim 12, wherein said tubulin isotype level is selected from the group consisting of tubulin isotype III and tubulin isotype IV.

14. The method of claim 12, wherein said biological sample is obtained less than 24 hours after treatment with the combretastatin.

15. The method of claim 12, wherein said biological sample is obtained less than 6 hours after treatment with the combretastatin.
